(19) United States
(12) Patent Application Publication
(10) Pub. No.: US 2022/0187289 A1
(43) Pub. Date: Jun. 16, 2022

(54) METHODS FOR DETECTING, ISOLATION, AND QUANTIFYING AN ANALYTE IN A SAMPLE BASED ON COLLOIDAL SUSPENSION OF PLASMONIC METAL NANOPARTICLES

(71) Applicants: Muthukumaran Packirisamy, Pierrefonds (CA); Srinivas Bathini, Montreal (CA)

(72) Inventors: Muthukumaran Packirisamy, Pierrefonds (CA); Srinivas Bathini, Montreal (CA)

(21) Appl. No.: 17/489,542

(22) Filed: Mar. 2, 2022

Related U.S. Application Data
(60) Provisional application No. 63/084,960, filed on Sep. 29, 2020.

(51) Int. Cl. G01N 33/543; G01N 33/552; G01N 33/553; G01N 33/58; B01L 3/00

(52) U.S. Cl. CPC G01N 33/54346; G01N 33/552; G01N 33/553; G01N 33/587; B01L 2200/16; B01L 2200/0621; B01L 2300/087; B01L 2400/0457; B01L 3/502715

(57) ABSTRACT

There are provided methods for quantifying an analyte in a sample, diagnosing a condition characterized by an excess or a depletion of an analyte in a biological sample, isolating analyte from a sample, and detecting an analyte in a sample. These method comprise the steps of providing a colloidal suspension of nanoparticles of a plasmonic metal, the nanoparticles having attached on their surface a binding moiety for selective attachment of said analyte and adding the sample to the suspension, thus producing a mixture in which said analyte is attached to the nanoparticles in suspension. Then, the methods further comprise the steps of either
allowing sedimentation of the nanoparticles with bound analyte, thereby producing a sediment comprising the nanoparticles with bound analyte and a supernatant, and measuring the Localized Surface Plasmon Resonance (LSPR) spectrum of the supernatant and/or recovering the sediment, or
measuring the Localized Surface Plasmon Resonance (LSPR) spectrum of the mixture.

### CROSS REFERENCE TO RELATED APPLICATIONS

N/A

### FIELD OF THE INVENTION

The present invention relates to method for the detection, isolation and quantification of an analyte in a sample. More specifically, the present invention is concerned with method using a colloidal suspension of nanoparticles of a plasmonic metal having attached on their surface a binding moiety for selective attachment of said analyte.

### BACKGROUND OF THE INVENTION

### Extracellular Vesicles (EVs)

Extracellular vesicles (EVs), known also as shedding vesicles or oncosomes, are vital sources of biomarkers for cancer and other pathological conditions such as inflammatory and neurodegenerative diseases and also for clinical diagnostics. They are membrane bounded nanoscale extracellular communication organelles that are released from almost all cell types to the extracellular space, transporting a cargo of active molecules (DNA, RNA, proteins/enzymes, lipid, and metabolites) reflecting the identity of their mother cells to neighboring and distal parts of the body. Therefore, they represent real-time snapshots of the physiological/pathological status of the mother cells. They are present in all body fluids, including urine, blood, ascites, and cerebrospinal fluid fractions of body fluids such as serum and plasma, as well as in cultured medium of cells.

Exosomes are a specific type of EVs, which are cup-shaped with a diameter ranging from 30 nm to 100 nm, which is about hundred times smaller than the smallest cell. Generally, they are released by inward budding of endosome membranes, followed by splitting of plasma membrane through the endocytic pathway. Exosomes carry membrane proteins and heat shock proteins such as HSP70.

When compared with circulating tumor cells (CTCs) and cell-free circulating tumor DNA (ctDNA), EVs/exosomes have lot of advantages in terms of stability, quantity and most importantly accessibility. That is, EVs/exosomes are abundantly released from cancer cells and are capable of protecting proteins and nucleic acids that are related to cancer development. In addition, they are very accessible because of their broad distribution in body fluids.

EVs (including exosomes) are thus potential sources for liquid biopsy, a remote and minimally invasive technique for early stage diagnosis of cancer and other diseases. Given the growing evidence that exosomes may be a clinically relevant biomarker source, there is a great demand for their simple and efficient detection in bio-fluids. Most affinity-based EV detection methods rely on antibodies directed against EV surface marker(s).

The detection, isolation, and characterization of exosomes are however still challenging due to the natural complexity of body fluids. For this reason, a versatile platform and an easy-to-use technique are required to adequately and selectively detect, isolate, quantify, and characterize exosomes for clinical applications.

The most widely used methods for separation of EVs/exosomes are ultracentrifugation, density gradient centrifugation, size exclusion chromatography, and immune based separation. Characterization techniques include electron microscopy, nanoparticle tracking analysis, flow cytometry and western blotting.

Currently available exosome isolation methods that are precipitation based (ultracentrifugation and using polyethylene glycol), are not suitable for point-of-care (POC) clinical-diagnosis. Ultrafiltration yields relatively pure exosomes but is technically challenging. For the development of routine exosome-based POC diagnostics, affinity-based exosome capture is technologically desirable. Most affinity-based exosome capture methods rely on monoclonal antibodies, directed against exosomes surface markers, driving higher the cost of production and inconsistency in assays such as batch-to-batch variations of antibodies.

Presently, the standard method for isolation of EVs from body fluids is differential ultracentrifugation, based on their physical characteristics. This is usually followed by ELISA (enzyme-linked immunosorbent assay) or western blot. However, both techniques are time consuming, requires large number of exosomes and results in low yield and therefore, not suitable for clinical applications.

Antibody-based affinity-capture and all the other precipitation-based exosome isolation methods facilitate the capture of all the exosomes present in the given fluid, without differentiating between healthy and diseased (cancer) exosomes. Quite sophisticated micro- and nano-systems have gained attention in recent years for their high sensitivity to detect exosomes. Among them are the electrochemistry-based approaches, using electroactive molecules tagged with a detection antibody and the detection of captured exosomes by electrochemical sensing. Nanoplasmonic sensors and microfluidic exosome analysis platforms having an antibody functionalized channel have also been reported.

In spite of the advances in exosome detection techniques, because of the complexity and heterogeneity of exosomes' composition, none of the existing techniques can be considered as a general method to be used for the detection of exosomes for both clinical purposes and research. There is still a lot of room for improvement.

Furthermore, it is evident in many cancers (for example, breast and ovarian) that the concentrations of the total cancer-cells and their exosome-bound HSPs are elevated and implicated in various aspects of cancer biology. In fact, cancer cells and their exosomes over express HSPs in their lumen as well as on their surface, but only at a minimum level in healthy cells or their exosomes. Also, cancerous cells release a higher number of exosomes, compared to normal cells implicated in tumor progression.

Thus, while it is important to characterize exosomes by their molecular composition, there is presently a growing demand for an accurate method to quantify the absolute concentration of exosomes in body fluids for potential POC diagnosis. For this purpose, surface-based detection methods such as Surface Plasmon Resonance (SPR) and, more recently, Localized Surface Plasmon Resonance (LSPR) have emerged, in addition to flow cytometry, Tunable Resistive Pulse Sensing (TRPS), and Nanoparticle Tracking Analysis (NTA).

### Bovine Growth Hormone (BGH)

On another subject, bovine Growth Hormone (BGH) is a natural growth hormone produced by the anterior pituitary glands in mammals. It is also known as bovine somatotropin (BST). Recombinant Bovine Growth Hormone (rBGH) is a synthetic hormone, which is used to increase the milk production in cows. It has been approved by in the United States but is not permitted in the European Union, Canada and some other countries. The use of rBGH is controversial because of its potential effects on animal and human health.

Conventionally, the methods for estimation of rBGH concentration were either radioimmunoassay (RIA) or enzyme-linked immunosorbent assay (ELISA) or bioassay. Recently, a method based on liquid chromatography-mass spectrometry combined with electrospray ionization has been developed, which allows the discrimination of recombinant from the endogenous forms of somatotropin. But the principal drawback of this approach is the complex methodology with very expensive instrumentation, makes it difficult for a rapid detection. Later on, a surface plasmon resonance (SPR) biosensing method has been suggested. However, the measurement was carried out in Biocore 3000, which is an expensive plasmonic instrument. Therefore, it is extremely desirable that, a highly sensitive methods that allows rapid and precise detection of growth hormones in milk are required to provide meaningful information about the rBGH-treated animals to the consumers.

### SUMMARY OF THE INVENTION

In accordance with the present invention, there is provided:
1. A method of quantifying an analyte in a sample, the method comprising the steps of:
   a) providing a colloidal suspension of nanoparticles of a plasmonic metal, the nanoparticles having attached on their surface a binding moiety for selective attachment of said analyte,
   b) adding the sample to the suspension, thus producing a mixture in which said analyte is attached to the nanoparticles in suspension,
   c) allowing sedimentation of the nanoparticles with bound analyte, thereby producing a sediment comprising the nanoparticles with bound analyte and a supernatant, and
   d) measuring the Localized Surface Plasmon Resonance (LSPR) spectrum of the supernatant,
      wherein the colloidal suspension comprises a known concentration of nanoparticles which can bind a known cut-off concentration of the analyte,
      wherein the presence of a plasmonic metal band in the LSPR spectrum of the supernatant indicates the sample had a concentration of the analyte lower than said cut-off concentration,
      wherein the presence of band associated with the analyte in the LSPR spectrum of the supernatant indicates that the sample had a concentration of the analyte higher than said cut-off concentration, and
      wherein the analyte is a biomolecule or a vesicle.
2. A method of diagnosing a condition characterized by an excess or a depletion of an analyte in a biological sample, the method comprising steps of:
   a) providing a colloidal suspension of nanoparticles of a plasmonic metal, the nanoparticles having attached on their surface a binding moiety for selective attachment of said analyte,
   b) adding the sample to the suspension, thus producing a mixture in which said analyte is attached to the nanoparticles in suspension,
   c) allowing sedimentation of the nanoparticles with bound analyte, thereby producing a sediment comprising the nanoparticles with bound analyte and a supernatant, and
   d) measuring the Localized Surface Plasmon Resonance (LSPR) spectrum of the supernatant,
      wherein the colloidal suspension comprises a known concentration of nanoparticles which can bind a known cut-off concentration of the analyte, wherein said cut-off concentration is between the average concentration of the analyte found in healthy subjects and the average concentration of the analyte found in subjects suffering from said condition,
      wherein the presence of a plasmonic metal band in the LSPR spectrum of the supernatant indicates that the sample had a concentration of the analyte lower than said cut-off concentration,
      wherein the presence of band associated with the analyte in the LSPR spectrum of the supernatant indicates that the sample had a concentration of the analyte higher than said cut-off concentration, and
      wherein the analyte is a biomolecule or a vesicle.
3. The method of item 1 or 2, wherein centrifugation is used at step c) to speed up the sedimentation of the nanoparticles.
4. A method of isolating analyte from a sample, the method comprising the steps of:
   a) providing a colloidal suspension of nanoparticles of a plasmonic metal, the nanoparticles having attached on their surface a binding moiety for selective attachment of said analyte;
   b) adding the sample to the suspension, thus producing a mixture in which said analyte is attached to the nanoparticles in suspension;
   c) allowing sedimentation of the nanoparticles with bound analyte, thereby producing a sediment comprising the nanoparticles with bound analyte and a supernatant;
   d′) recovering the sediment,
      wherein the analyte is a biomolecule or a vesicle.
5. The method of item 4, further comprising the steps of:
   optionally, recovering the analyte form the sediment, and
   carrying out molecular analysis on the sediment or on the separated analyte.
6. A method of detecting an analyte in a sample, the method comprising the steps of:
   a) providing a colloidal suspension of nanoparticles of a plasmonic metal, the nanoparticles having attached on their surface a binding moiety for selective attachment of said analyte;
   b) adding the sample to the suspension thus producing a mixture in which said analyte is attached to the nanoparticles in suspension; and
   c′) measuring the Localized Surface Plasmon Resonance (LSPR) spectrum of the mixture,
      wherein a shift in the position of the plasmonic metal band in the LSPR spectrum of the mixture compared to that of the suspension before addition of the sample indicates the presence of said analyte in the sample, and
      wherein the analyte is a biomolecule or a vesicle.
7. The method of any one of items 1 to 6, wherein the analyte is a biomolecule or a vesicle, preferably a vesicle, and more preferably an exosome.
8. The method of any one of items 1 to 7, wherein the sample is a biological sample, preferably a biofluids.
9. The method of any one of items 1 to 8, further comprise, before step a), a step of preparing nanoparticles of a plasmonic metal, the nanoparticles having attached on their surface a binding moiety for attachment of said analyte.
10. The method of any one of items 1 to 9, wherein the binding moiety is attached to a linker, which is attached to the nanoparticle surface.
11. A device for performing the method of any one of items 1 to 10, the device comprising:
   a) a suspension inlet (12) for introducing a colloidal suspension of nanoparticles of a plasmonic metal, the nanoparticles having attached on their surface a binding moiety for selective attachment of said analyte,
   b) a sample inlet (14) for introducing the sample into the device,
   c) a mixing chamber (16) for mixing the sample with the suspension, thus producing a mixture in which said analyte is attached to the nanoparticles in suspension, and
   d) a sedimentation network (18) to allow sedimentation of the nanoparticles with bound analyte, thereby producing a sediment comprising the nanoparticles with bound analyte and a supernatant,
      wherein the sedimentation network (18) comprises a sediment container (20) and a supernatant container (22).
12. The device of item 11, wherein the sedimentation network (18) is gravity-assisted.
13. The device of item 11 or 12, wherein the sedimentation network (18) comprises a serpentine microfluidic channel.
14. The device of any one of items 11 to 13, wherein the supernatant container (22) comprises a sediment outlet (24) to allow recovery of the sediment.
15. The device of any one of items 11 to 14, wherein the sedimentation network (18) comprises a supernatant outlet (26) to allow recovery of the supernatant for LSPR analysis.
16. The device of any one of items 11 to 14, wherein the supernatant container (22) functions as a cuvette allowing recording the LSPR spectrum while the supernatant is held in the device.
17. The device of any one of items 11 to 16, wherein the device (10) is a hand-held device.
18. The device of any one of items 11 to 16, wherein the device (10) is a macroscale device, a microscale device, or in a microfluidic device.
19. The device of any one of items 15 to 18, wherein the device (10) is a point-of-care testing device e.g. for quantifying an analyte in a sample and/or diagnosing a condition characterized by an excess or a depletion of an analyte in a biological sample, e.g. cancer.
20. The device of any one of items 15 to 19, wherein the device (10) is for rBGH detection.
21. The device of any one of items 15 to 20, wherein the device (10) comprises multiple isolation units (i.e. inlets (12, 14), mixing chamber (16), and sedimentation network (18)) allowing the detection of analyte in various samples.

### BRIEF DESCRIPTION OF THE DRAWINGS

In the appended drawings:

FIG. 1 LSPR absorption spectra at each stage of the biosensing protocol of Example 2.

FIG. 2 LSPR absorption spectra at each stage of the biosensing protocol of Example 3.

FIG. 3 Evolution of the LSPR shift with reaction time after ECD+NHS addition.

FIG. 4 Evolution of the LSPR shift with reaction time after BGH antibody addition.

FIG. 5 Biosensing protocol for the plasmonic detection of exosomes in Example 4.

FIG. 6 Shows details of steps e″ and f″ of the biosensing protocol of Example 4.

FIG. 7 LSPR absorption spectra recorded at each stage of the biosensing protocol of Example 4.

FIG. 8 LSPR absorption spectra of supernatant after 8 hours of biosensing protocol.

FIG. 9 LSPR absorption spectra of supernatant after centrifugation at 10000 g for 2 minutes.

FIG. 10 LSPR absorption spectra of gold colloidal suspension prepared using 18 mg, 23 mg or 30 mg of gold precursor (HAuCl₄.3H₂O).

FIG. 11 Gold concentration of the undiluted and diluted suspensions as prepared in Example 4B as function of their LSPR gold band absorbance.

FIG. 12 Absorption spectra of supernatant obtained using the diluted GCSs prepared using 23 mg of precursor.

FIG. 13 Absorption spectra of supernatant obtained using the diluted GCSs prepared using 18 mg of precursor.

FIG. 14 Absorption spectra of supernatant obtained using the diluted GCSs prepared using 30 mg of precursor.

FIG. 15 Cross-sectional view of a gold nanoparticle, with successive layers of bio-entities immobilized thereon, accommodating a number of exosomes.

FIG. 16 Predicted gold concentration required to remove all the exosomes of a representative cancerous sample (labelled “Undiluted”) and this sample diluted by a factor of 2, 5, 10, or 50 as a function of AuNPs size.

FIG. 17 ¾ % view of a microfluidic device according to an embodiment of the invention.

### DETAILED DESCRIPTION OF THE INVENTION

Turning now to the invention in more details, there are provided methods for the detection, isolation and quantification of a biomolecule/vesicle in a sample as well as a method of diagnosing a condition characterized by an excess or a depletion of a biomolecule/vesicle in a biological sample. All these methods are based on the use of a colloidal suspension of nanoparticles of a plasmonic metal, which have attached on their surface a binding moiety for selective attachment of said biomolecule/vesicle.

In a first aspect of the invention, there is thus provided a method of quantifying an analyte in a sample, the method comprising the steps of:
a) providing a colloidal suspension of nanoparticles of a plasmonic metal, the nanoparticles having attached on their surface a binding moiety for selective attachment of said analyte,
b) adding the sample to the suspension, thus producing a mixture in which said analyte is attached to the nanoparticles in suspension,
c) allowing sedimentation of the nanoparticles with bound analyte, thereby producing a sediment comprising the nanoparticles with bound analyte and a supernatant, and
d) measuring the Localized Surface Plasmon Resonance (LSPR) spectrum of the supernatant,
   wherein the colloidal suspension comprises a known concentration of nanoparticles which can bind a known cut-off concentration of the analyte,
   wherein the presence of a plasmonic metal band in the LSPR spectrum of the supernatant indicates the sample had a concentration of the analyte lower than said cut-off concentration,
   wherein the presence of band associated with the analyte in the LSPR spectrum of the supernatant indicates that the sample had a concentration of the analyte higher than said cut-off concentration, and
   wherein the analyte is a biomolecule or a vesicle.

In a second aspect of the invention, there is thus provided a method of diagnosing a condition characterized by an excess or a depletion of an analyte in a biological sample, the method comprising steps of:
a) providing a colloidal suspension of nanoparticles of a plasmonic metal, the nanoparticles having attached on their surface a binding moiety for selective attachment of said analyte,
b) adding the sample to the suspension, thus producing a mixture in which said analyte is attached to the nanoparticles in suspension,
c) allowing sedimentation of the nanoparticles with bound analyte, thereby producing a sediment comprising the nanoparticles with bound analyte and a supernatant, and
d) measuring the Localized Surface Plasmon Resonance (LSPR) spectrum of the supernatant,
   wherein the colloidal suspension comprises a known concentration of nanoparticles which can bind a known cut-off concentration of the analyte, wherein said cut-off concentration is between the average concentration of the analyte found in healthy subjects and the average concentration of the analyte found in subjects suffering from said condition,
   wherein the presence of a plasmonic metal band in the LSPR spectrum of the supernatant indicates that the sample had a concentration of the analyte lower than said cut-off concentration,
   wherein the presence of band associated with the analyte in the LSPR spectrum of the supernatant indicates that the sample had a concentration of the analyte higher than said cut-off concentration, and
   wherein the analyte is a biomolecule or a vesicle.

In a third aspect of the invention, there is thus provided a method of isolating analyte from a sample, the method comprising the steps of:
a) providing a colloidal suspension of nanoparticles of a plasmonic metal, the nanoparticles having attached on their surface a binding moiety for selective attachment of said analyte;
b) adding the sample to the suspension, thus producing a mixture in which said analyte is attached to the nanoparticles in suspension;
c) allowing sedimentation of the nanoparticles with bound analyte, thereby producing a sediment comprising the nanoparticles with bound analyte and a supernatant;
d′) recovering the sediment,
   wherein the analyte is a biomolecule or a vesicle.

In a fourth aspect of the invention, there is thus provided a method of detecting an analyte in a sample, the method comprising the steps of:
a) providing a colloidal suspension of nanoparticles of a plasmonic metal, the nanoparticles having attached on, their surface a binding moiety for selective attachment of said analyte;
b) adding the sample to the suspension thus producing a mixture in which said analyte is attached to the nanoparticles in suspension; and
c′) measuring the Localized Surface Plasmon Resonance (LSPR) spectrum of the mixture,
   wherein a shift in the position of the plasmonic metal band in the LSPR spectrum of the mixture compared to that of the suspension before addition of the sample indicates the presence of said analyte in the sample, and
   wherein the analyte is a biomolecule or a vesicle.

The above methods have many advantages. As can be seen from the above four aspects to the invention, the present “colloidal platform” is multi-functional allowing selectively detecting, isolating, and quantifying analytes.

The methods of the invention can be applied to various fields and analytes, for example environmental contaminants, pathogens, as well as the diagnosis of diseases. In particular, they can be applied to EVs/exosomes (e.g. as biomarkers of cancer) as well as proteins and hormones (such as rBGH e.g. in milk). Turning now specifically to EVs/exosomes, it should be noted that conventional detection and isolation techniques are slow and requires large numbers of exosomes. Further, the exosomes are isolation with a yield too low to be clinically useful. The methods of the invention only require low-volume samples (microliters) and are fast.

The plasmonic detection is based on the sensitivity of the LSPR spectrum of plasmonic metal nanoparticles. The proposed method replaces the plasmonic sensing in solid state where multilayers of plasmonic metal are immobilized on a solid substrate. This difference is very advantageous in various unexpected ways:
The sensitivity is much higher.
Reaction times are significantly faster (detection, isolation).
The analyte can effectively be isolated.

Further, by varying the size and concentration of the plasmonic metal nanoparticles, the cut-off concentration above which the suspension can no longer bind and remove the analyte by sedimentation can be controlled. This allows quantifying the analyte. This also allows discriminating between two states e.g. a diseased state and a healthy state, by using a suspension with a cut-off concentration between the analyte concentration characterizing these two states. In turns, this allows the non-invasive diagnostic of conditions characterized by an excess or a depletion of an analyte in a biological sample as well as the monitoring of a course of treatment, the efficacy of which would be reflected by a change in the concentration of an analyte in a biological sample.

The method according to the fourth aspect of the invention, based on the interaction of an analyte with a binding moiety on a plasmonic metal nanoparticle, allows the quick and easy detection of this interaction, which a high sensitivity and at a low price, using the position of the plasmonic metal band in LSPR spectrum of the mixture.

Further, the methods of the invention can be translated into a fast and cheap sensor with high sensitivity (compared to conventional SPR methods).

### Analytes and Samples

The above methods allow to detect, quantify, or isolate an analyte in a sample or to diagnose a condition characterized by an excess or a depletion of an analyte in a biological sample. Herein, an “analyte” is a substance in a sample that is of interest in an analytical/isolation procedure such as the methods of the invention.

As noted above, the analyte is a biomolecule or a vesicle. Preferably, the analyte is a vesicle, and more preferably an exosome.

A biomolecule is a molecule from a biological organism that is involved in one or more biological processes in said organism. Preferred biomolecules that can be analytes in the methods of the invention are macromolecules, which are large molecules composed repeating monomers. Non-limiting examples of biomolecules that can be analytes in the methods of the invention include nucleic acids as well as proteins, such as hormones, preferably growth hormones, more preferably bovine growth hormone (BGH), either native or recombinant, and most preferably recombinant bovine growth hormone (rBGH).

A vesicle is a structure, typically present within or outside the cells of a biological organism, comprising a cytoplasm enclosed by a lipid bilayer. Non-limiting examples of vesicles that can be analytes in the methods of the invention include extracellular vesicles, such as exosomes, preferably exosomes from diseased cells, such as e.g. cancerous cells. In such cases, the methods of the invention allow for the detection of cancers.

The sample in the above methods can be any sample, preferably it is a biological sample (i.e. a sample obtained from an organism), more preferably it is a biofluid. Non-limiting examples of biofluids include those that are excreted (such as urine or sweat), are secreted (such as breast milk or bile), are obtained with a needle (such as blood or cerebrospinal fluid), or have developed as a result of a pathological process (such as blister or cyst fluid).

### Analyte/Binding Moiety Attachment

In the methods of the invention, the nanoparticles have attached on their surface a binding moiety for selective attachment of the analyte and consequently when the nanoparticles and the analyte come into contact (at step b of each of the methods of the invention), the analyte becomes attached to the nanoparticles in suspension. At the same time, any other component from the sample does not attach to the nanoparticles.

The attachment of the binding moiety to the analyte can occur in various ways. In fact, any binding moiety with a high affinity and selectivity towards the analyte can be used. Well-known antigen/antibody and peptide/protein affinity interactions can be used. For example:
The binding can be any antigen-antibody type biospecific binding. In such cases, the binding moiety can be one of an antigen or an antibody and the analyte can be the other.
   For example, a BGH antigen can be used as a binding moiety when the analyte is BGH or rBGH.
The binding can be any protein and anti-protein type biospecific binding.
   For example, the so-called Vn96 peptide has a high specificity and affinity for canonical heat shock proteins (HSPs) lining the surface of exosomes and other EVs. Thus, the Vn96 peptide and other antibodies such as CD63, CD9, CD81 can be used as the binding moiety when the analyte is an exosome or another EV.
   Avidin/streptavidin binds to biotin with a high degree of affinity and specificity. This interaction can be exploited with the binding moiety being one of the avidin/streptavidin or the biotin and the analyte being the other.

Importantly, when there are no known potential binding moieties for a given analyte, prior to step b of the above method, the analyte in the sample can be specifically tagged with (i.e. have grafted thereon) a moiety that will become bound to the binding moiety during step b). For example, an analyte can be tagged with either biotin or avidin/streptavidin (preferably biotin) so it can become bound to the binding moiety (which would be the other of biotin or avidin/streptavidin).

It will be apparent to the skilled person that the nanoparticles preferably bear on their surface multiple copies of a same binding moieties to bind several copies of the analytes.

In embodiments, the nanoparticles can bear on their two or more different binding moieties, each selectively binding to different analytes.

### Nanoparticles

In embodiments, the methods of the invention comprise, before step a), a step of preparing nanoparticles of a plasmonic metal, the nanoparticles having attached on their surface a binding moiety for attachment of said analyte.

The nanoparticles of a plasmonic metal can be made of any such plasmonic metal and their alloys or mixtures, preferably they are made of a plasmonic metal (not an alloy or mixture), more preferably silver or gold, preferably gold. The nanoparticles are typically between about 10 nm and 120 nm in size, preferably between about 10 nm and 80 nm in size. The nanoparticles can be of any shape, e.g. spheroidal, ellipsoidal, irregularly shaped, rod-shaped, discoidal, star-shape, etc. Preferably, the nanoparticles are spheroidal.

The nanoparticles can be prepared by any method known to the skilled person. For example, Turkevich et al. (Turkevich, J.; Stevenson, P. C.; Hillier, J. Discuss. Faraday Soc. 1951, 11, 55; Enu\ddot{ }stu\ddot{ }n, B. V.; Turkevich, J. J. Am. Chem. Soc. 1963, 85, 3317 and J. Kimling, M. Maier, B. Okenve, V. Kotaidis, H. Ballot, and A. Plech, “Turkevich Method for Gold Nanoparticle Synthesis Revisited”, J. Phys. Chem. B 2006, 110, 15700-15707) taught a method for the preparation of spheroidal gold nanoparticles by reducing a gold precursor, hydrogen tetrachloroaurate (HAuCl₄), with sodium citrate in boiling water. This method allows to control the diameter of the nanoparticles as desired.

The binding moiety can be attached to the surface of the plasmonic metal particle by any means known to the skilled person. In particular, thiol chemistry is well-known for attaching molecules to gold or other metal nano surfaces such as silver nanoparticles and magnetic beads. These molecules can then be used as handles to attach the binding moieties.

In embodiments, the binding moiety is attached to a linker, which is attached to the nanoparticle surface. This linker can have two related roles: holding the biding moiety further from the nanoparticle surface, which can allow more analyte (especially vesicles) to ultimately become attached to the nanoparticles at step b—see Example 4C; and easing sedimentation of the nanoparticles once the analyte is bound to the nanoparticles. Sedimentation is preferable since it leads to nanoparticles that can be used in all the methods of the invention. However, even when no sedimentation occurs, the nanoparticles can still be used in the method for detecting an analyte as provided in the fourth aspect of the invention.

Thiols that can be used in the above including compounds of formula SH—R—COOH, wherein R is a linear or branched, preferably linear, alkylene, alkenylene, alkynylene, or alkenynylene. An example of a thiol molecule that can attach itself to gold and yield a useful chemical handle is 11-mercaptoundecanoic acid (e.g. NanoThinks® ACID11) and 16-mercaptohexdecanoic acid (e.g. NanoThinks® ACID16). In these cases, the chemical handle is a carboxyl (—COOH) group terminating the alkylene chain. This molecule also has the added benefit of stabilizing the nanoparticles in the suspension (i.e. preventing their unwanted aggregation).

Au+S—R—COOH→Au—S—R—COOH

wherein “Au” denotes the surface of the gold nanoparticle.

Advantageously, after attachment, the above carboxyl group can be activated, for example using a compound containing N-(3-dimethylaminopropyl)-N′-ethylcarbodiimide hydrochloride (EDC) and N-hydroxysuccinimide (NHS). These yield an amine-reactive sulfo-NHS ester that is reactive towards primary amines, which are commonly found in the binding moieties described above. The reaction of this amine-reactive sulfo-NHS ester with an amine ultimately forms an amide bond as shown below.

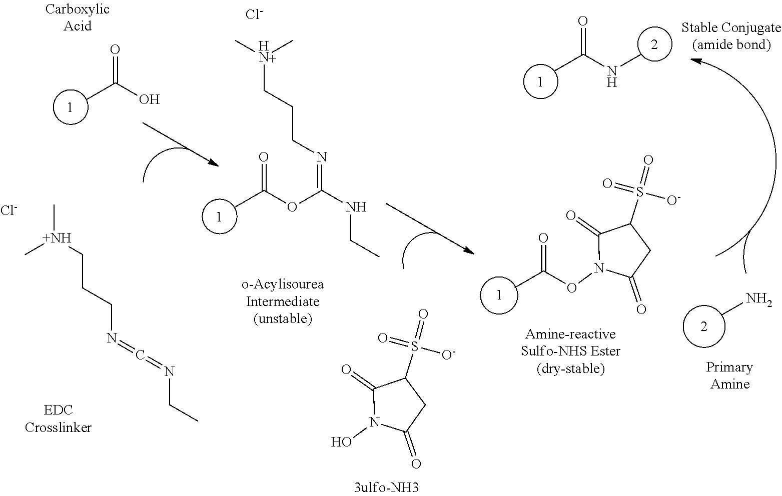

Thus, in embodiments, binding moieties such as antigens, antibodies, peptides and proteins, which typically comprise primary amines, can become directly attached to the nanoparticles through reaction of their amine with the activated carboxyl group (i.e. the amine-reactive sulfo-NHS ester).

Au—S—R—COOH (preferably activated)+binding moiety with primary amine group→Au—S—R—CO—(NH-binding moiety)

wherein “Au” denotes the surface of the gold nanoparticle and “NH-binding moiety” denotes a binding moiety that has become attached through reaction of one of its primary amine groups.

In other embodiments, a spacer is attached via reaction with the activated carboxyl group and the binding moiety is attached to this spacer. In embodiments, the linker is a streptavidin (or avidin)-biotin couple attached to a linker.

Au—S—R—CO—(NH-avidin)-biotin-linker-binding moiety

In such cases, the streptavidin (or avidin)-biotin couple is used to ease attachment of the spacer to the activated carboxyl group and the binding moiety is attached to the spacer. This arrangement can be prepared by attaching avidin (or streptavidin) to the activated carboxyl group (as described above when attaching a binding moiety), then reacting the nanoparticles with a biotin-linker-binding moiety compound. The linker can be e.g. linear or branched (preferably linear) alkylene, alkenylene, alkynylene, or alkenynylene, optionally terminated at either or both ends with residues of functional groups (such as amine and carboxyl groups) that allowed the linker to become bound to the biotin and the binding moiety.

### Colloidal Suspension & Sedimentation

Steps a) and b) of the methods of the invention involve a colloidal suspension of nanoparticles. Herein, a colloidal suspension a heterogenous mixture that contains solid particles (in the present case nanoparticles) dispersed and floating around freely in a liquid. The particles do not dissolve in the liquid. The particles do not settle or take a very long time to settle appreciably. This is in contrast with non-colloidal suspensions which settle much more quickly.

At step c) of the methods according to the first, second, and third aspect of the invention, after being bound to the analyte, the nanoparticles are allowed to sediment. Sedimentation is the settling of particles out of a liquid in which they are suspended, which causes them to come to rest against a barrier (for example the bottom of a container). Sedimentation result in a sediment comprising the settled particles and a supernatant devoid of these particles. Thus, in embodiments of step c) of the above methods, the nanoparticles with bound analyte are allowed to spontaneously settle under the action of gravity. This step can take between about 45 min and about 4 hours, preferably between about 45 min and about 2 hours.

In alternative embodiments of step c) of the above methods, centrifugation is used to speed up the sedimentation of the nanoparticles. Of course, ultracentrifugation, which would also cause the sedimentation of the unbound exosome and other materials, should preferably be avoided in this step. As explained in the next two sections, it is a significant advantage of the invention that only the nanoparticles with bound analyte settle while all other entities including unbound nanoparticles, unbound analyte, and other components of the sample remain in suspension (or solution as the case may be).

### Isolation of the Analyte—Method According to the 3^{rd} Aspect of the Invention

The selective sedimentation of nanoparticles with bound analyte allows to selectively isolate the analyte from unbound nanoparticles and other sample components—simply by recovering the sediment as recited in the method according to the third aspect of the invention.

This isolation is quite rapid (occurring on the same timescale as the sedimentation), i.e. between about 45 min and about 4 hours, preferably between about 45 min and about 2 hours. In comparison, isolation using a conventional ultracentrifugation technique may take anywhere from 4 to 6 hours.

Further, the sedimentation rate can be optimized by optimizing the concentration and size of the nanoparticles in the suspension. Indeed, gold nanoparticles of a certain size can only accommodate a certain number of copies of an analyte (of a certain size). When the size and concentration of analyte in a sample are known or can be estimated, the gold concentration can be adjusted to have just enough nanoparticles to bind all the analyte or a slight excess. This ensure a maximum number of analyte copies bound to each nanoparticle and should thus speed the sedimentation.

In addition, as long as the suspension used contains enough nanoparticles to bind all the analyte, the method according to the third aspect of the invention allows for complete or near-complete isolation of the analyte.

In embodiments, the method according to the third aspect of the invention can further comprise the steps of:
optionally, recovering the analyte form the sediment, and
carrying out molecular analysis on the sediment or on the separated analyte.
   This can be done, inter alias, for diagnostic purposes.

### Quantification/Diagnostic—Method According to the 1^{st} and 2^{nd} Aspects of the Invention

The selective sedimentation of nanoparticles with bound analyte allows the quantification of the analyte and thus the eventual diagnostic of a condition characterized by an excess or a depletion of an analyte in a biological sample as per the methods according to the first and second aspects of the invention.

Indeed, gold nanoparticles of a given size can only accommodate a certain number of copies of an analyte of a given size. Knowing the number of copies of an analyte that one nanoparticle can accommodate and the number of nanoparticles in the suspension (calculated from the nanoparticles concentration, size and density), it is possible to calculate the maximum quantity of analyte that will be removed during the sedimentation step—see example 4C for more details on these calculations. In other words, each suspension has a cut-off concentration above which it can no longer bind and remove the analyte by sedimentation (which would leave unbound analyte in the supernatant). On the other hand, if the analyte concentration was below this cut-off concentration, unbound nanoparticles will remain in the supernatant. Thus, by analyzing the composition of the supernatant (by LSPR as described below) looking for unbound analyte and unbound nanoparticles, one can determine whether the analyte concentration was above or below the cut-off concentration.

When one desires to use the methods according to the first and second aspects of the invention to discriminate between two states (e.g. a healthy and a diseased states), it is particularly advantageous to tune the nanoparticles size and/or nanoparticle concentration in the suspension so that the cut-off concentration lies between the typical analyte concentration encountered in these two states. This is why, in the method according to the second aspect of the invention, the cut-off concentration is between the average concentration of the analyte found in healthy subjects and the average concentration of the analyte found in subjects suffering from the condition probed for. The exosome concentration is increased in patients with cancer (up to 50 times that of a cancer-free individual). The methods of the invention would thus allow to detect cancer, even in its early stages.

### LSPR Spectroscopy

Localized surface plasmon resonance (LSPR) spectroscopy of plasmonic metal (typically gold or silver) nanoparticles is a powerful technique for chemical and biological sensing experiments.

LSPR is one of the most important optical properties of plasmonic metal nanoparticles and nanostructures. It occurs when the oscillation of free conduction electrons of nanoparticles is resonant with the incident light. Both the position and the intensity of LSPR band are dependent on the size and shape of nanoparticles and they are highly sensitive to dielectric properties of the surrounding medium. In particular, the position and the intensity of LSPR can shift with various moieties are attached on the plasmonic metal nanoparticles.

### Quantification/Diagnostic—Method According to the 1^{st} and 2^{nd} Aspects of the Invention

As noted above, each suspension has a cut-off concentration above which it can no longer bind and remove an analyte by sedimentation (which would leave unbound analyte in the supernatant). However, if the analyte concentration is below this cut-off concentration, unbound nanoparticles will remain in the supernatant.

Thus, step d) of the methods according to the first and second aspects of the invention, the LSPR spectrum of the supernatant is measured. This allows to analyze the composition of the supernatant and determine whether it contains unbound analyte or unbound nanoparticles. In turns, this allows to conclude whether the analyte concentration in the sample was above or below the cut-off concentration.

Namely, the presence of a plasmonic metal band in the LSPR spectrum of the supernatant indicates the sample had a concentration of the analyte lower than said cut-off concentration. The band can be for example the gold band at about 570 nm.

Further, the presence of band associated with the analyte in the LSPR spectrum of the supernatant indicates that the sample had a concentration of the analyte higher than said cut-off concentration. This band can be, for example, a protein band at about 265 nm. Note that it is possible than components other than the analyte contribute to this band or other similar bands. In such cases, the LSPR spectrum of the supernatant under study can be corrected using a comparative LSPR spectrum of a supernatant in which all the analyte has been removed as the band observed in this comparative spectrum would entirely be due to components other than the analyte. A supernatant in which all the analyte has been removed can be obtained using an excess, even large excess, of gold nanoparticles so all the analyte is removed.

### Detection—Method According to the 4^{th} Aspect of the Invention

In step d′) of the method according to the fourth aspect of the invention, the LSPR spectrum of the mixture (that is the nanoparticle suspension mixed with sample) is recorded. This spectrum must be recorded in a mixture that does not sediment or before the mixture has had the time to significantly sediment. Spectrum can be typically measured within about one minute, but it is preferred to wait for about 10 minutes for complete binding. In such a mixture, one expects to see a band arising from the plasmonic metal making the nanoparticles (e.g. the gold band at about 570 nm).

The position of this band is of particular interest as it will shift when the analyte binds to the nanoparticles. In other words, a shift in the position of the plasmonic metal band in the LSPR spectrum of the mixture compared to that of the suspension before addition of the sample indicates the presence of the analyte in the mixture (and thus in the sample that was added to the suspension). Thus, this method allows to detect the presence of the analyte in the sample. Furthermore, the magnitude of the band shift could also be used to provide an estimate of the analyte concentration.

We show in Example 3 below, that this method allows to detect BGH with a sensitivity of <0.1 ng/ml. This very low detection limit means that various conditions can likely be detected at an early stage.

Further, the nanoparticle concentration can be tuned depending on the (expected) analyte concentration to have complete or almost complete binding of the analyte to the nanoparticles and avoid that any free analyte remains.

### Device

In a fifth aspect of the invention, there is provided a device for performing any one or more, preferably all, the methods of the invention.

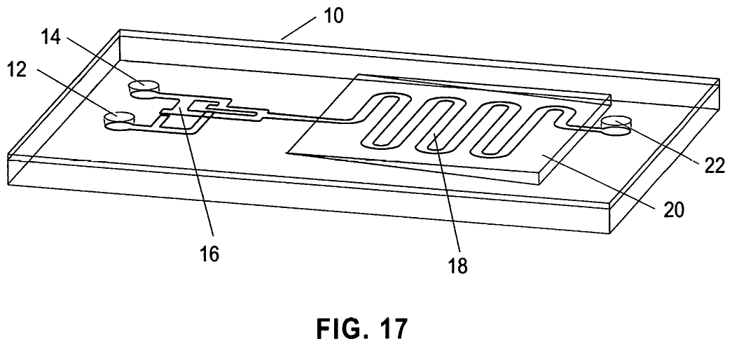

An example of such device is shown in FIG. 17.

This device (10) comprises:
a suspension inlet (12) for introducing a colloidal suspension of nanoparticles of a plasmonic metal, the nanoparticles having attached on their surface a binding moiety for selective attachment of said analyte,
a sample inlet (14) for introducing the sample into the device,
a mixing chamber (16) for mixing the sample with the suspension, thus producing a mixture in which said analyte is attached to the nanoparticles in suspension, and
a sedimentation network (18) to allow sedimentation of the nanoparticles with bound analyte, thereby producing a sediment comprising the nanoparticles with bound analyte and a supernatant,
   the sedimentation network (18) comprising a sediment container (20) and a supernatant container (22).

In preferred embodiments, the sedimentation network (18) is gravity-assisted.

In preferred embodiments, the sedimentation network (18) comprises a serpentine microfluidic channel. The number of turns in this serpentine will depend on the flowrate and the time required for sedimentation. As the mixture passes through the sedimentation network, the nanoparticles with bound analyte will settle due to gravity, while the supernatant will be left in the serpentine microfluidic channel.

In preferred embodiments, the supernatant container (22) comprises a sediment outlet (24), not shown, to allow recovery of the sediment.

In preferred embodiments, the sedimentation network (18) comprises a supernatant outlet (26), not shown, to allow recovery of the supernatant for LSPR analysis. Alternatively, in embodiments, the supernatant container (22) functions as a cuvette allowing recording the LSPR spectrum while the supernatant is held in the device.

In preferred embodiments, the device (10) is a hand-held device. It can be a macroscale, microscale, or microfluidic device. Preferably, it is a microfluidic device.

In embodiments, the device (10) is a point-of-care testing device e.g. for quantifying an analyte in a sample and/or diagnosing a condition characterized by an excess or a depletion of an analyte in a biological sample, e.g. cancer.

In embodiments, the device (10) is for rBGH detection.

In embodiments, the device (10) comprises multiple isolation units (i.e. inlets (12, 14), mixing chamber (16), and sedimentation network (18)) allowing the detection of analyte in various samples.

Further, the detection can be performed by using the device (10) with an appropriate spectrometer.

### Definitions

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted. In contrast, the phrase “consisting of” excludes any unspecified element, step, ingredient, or the like. The phrase “consisting essentially of” limits the scope to the specified materials or steps and those that do not materially affect the basic and novel characteristic(s) of the invention.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. In embodiments, it may mean plus or minus 10% or plus or minus 5% of the numerical value qualified.

Unless otherwise defined, all technical and scientific terms used herein have the same meaning as commonly understood by one of ordinary skill in the art to which this invention belongs.

Herein, the terms “alkyl”, “alkylene”, “alkenyl”, “alkenylene”, “alkynyl”, “alkynylene” and their derivatives (such as alkoxy, alkyleneoxy, etc.) have their ordinary meaning in the art. For more certainty, herein:

| | |
|---|---|
| Term | Definition |

Saturated aliphatic hydrocarbons

| | |
|---|---|
| alkane | aliphatic hydrocarbon of general formula CₙH₂ₙ₊₂ |
| alkyl | monovalent alkane radical of general formula |
| | —CₙH₂ₙ₊₁ |
| alkylene | bivalent alkane radical of general formula —CₙH₂ₙ— |
| (also called | |
| alkanediyl) | |

Aliphatic hydrocarbons with double bond(s)

| | |
|---|---|
| alkene | aliphatic hydrocarbon, similar to an alkane but |
| | comprising at least one double bond |
| alkenyl | monovalent alkene radical, similar to an alkyl but |
| | comprising at least one double bond |
| alkenylene | bivalent alkene radical, similar to an alkylene but |
| | comprising at least one double bond |

Aliphatic hydrocarbons with triple bond(s)

| | |
|---|---|
| alkyne | aliphatic hydrocarbon, similar to an alkane but |
| | comprising at least one triple bond |
| alkynyl | monovalent alkyne radical, similar to an alkyl but |
| | comprising at least one triple bond |
| alkynylene | bivalent alkyne radical, similar to an alkylene but |
| | comprising at least one triple bond |

Aliphatic hydrocarbons with double and triple bond(s)

| | |
|---|---|
| alkenyne | aliphatic hydrocarbon, similar to an alkane but |
| | comprising at least one double bond and at least |
| | one triple bond |
| alkenynyl | monovalent alkenyne radical, similar to an alkyl |
| | but comprising at least one double bond and at |
| | least one triple bond |
| alkenynylene | bivalent alkenyne radical, similar to an alkylene but |
| | comprising at least one double bond and at least |
| | one triple bond |

“Derivatives”

| | |
|---|---|
| alkyloxy or | monovalent radical of formula —O—alkyl |
| alkoxy | |
| alkyleneoxy | bivalent radical of formula —O—alkylene—. |
| | An example of alkyleneoxy is —O—CH₂—CH₂—, |
| | which is called ethyleneoxy. A linear chain |
| | comprising two or more ethyleneoxy groups attached |
| | together (i.e. —[O—CH₂—CH₂]ₙ—) can be referred |
| | to as a polyethylene glycol (PEG), polyethylene |
| | oxide (PEO), or polyoxyethylene (POE) chain. |
| alkenyloxy | monovalent radical of formula —O—alkenyl |
| alkenyleneoxy | bivalent radical of formula —O—alkenylene— |
| alkynyloxy | monovalent radical of formula —O—alkynyl |
| alkynyleneoxy | bivalent radical of formula —O—alkynylene— |

It is to be noted that, unless otherwise specified, the hydrocarbon chains of the above groups can be linear or branched. Further, unless otherwise specified, these groups can contain between 1 and 18 carbon atoms, more specifically between 1 and 12 carbon atoms, between 1 and 6 carbon atoms, between 1 and 3 carbon atoms, or contain 1 or 2, preferably 1, or preferably 2 carbon atoms.

Other objects, advantages and features of the present invention will become more apparent upon reading of the following non-restrictive description of specific embodiments thereof, given by way of example only with reference to the accompanying drawings.

### DESCRIPTION OF ILLUSTRATIVE EMBODIMENTS

The present invention is illustrated in further details by the following non-limiting examples.

### Example 1

### Materials & Methods and Preparation of a Gold Colloidal Suspension (GCS)

Gold (III) chloride trihydrate (HAuCl₄.3H₂O) and sodium citrate were purchased from Sigma Aldrich®. De-ionized (DI) water with a resistivity of 18 MΩ, used in all the experiments, was obtained by using a NANOpure® ultrapure water system (Barnstead®). A quartz cuvette with a 1 cm path length was purchased from Sigma Aldrich®. 11-mercaptoundecanoic acid in ethanol (NanoThinks® Acid 11), N-(3-Dimethylaminopropyl)-N′-ethylcarbodiimide hydrochloride (EDC), N-Hydroxysuccinimide (NHS), and streptavidin were purchased from IBA® GmBH. Biotin-PEG-Alexa647 was purchased from NANOCS®. Antibody BGH and BGH antigen were purchased from Cedarlane® labs. Vn96-linker-biotin and MCF-7 exosomes were supplied by the Atlantic Cancer Research Institute (ACRI), Moncton, New Brunswick, Canada. MCF-7 is a breast cancer cell line isolated in 1970 from a 69-year-old Caucasian woman. MCF-7 is the acronym of Michigan Cancer Foundation-7, referring to the institute in Detroit where the cell line was established in 1973.

Gold (III) chloride trihydrate (HAuCl₄.3H₂O) is a gold precursor used to prepare the gold colloidal suspension. 23 mg (Examples 2 and 3) or 18 mg, 23 mg and 30 mg (Example 4) of gold (III) chloride trihydrate (HAuCl₄.3H₂O) was added to 95 ml of DI water in a beaker and boiled till it reached its boiling point (around 10 minutes). Sodium citrate, a reducing agent to reduce to gold ions to gold nanoparticles, was prepared with a concentration of 2% using DI water. Once the reaction mixture reached its boiling point, 5 ml of the 2% sodium citrate solution were added. The reaction mixture was further boiled for about 5 to 8 minutes. A change in color to transparent purple was observed. Once the color change was observed, the suspension was allowed cool down to room temperature. DI water was added to attain a 100 ml final volume.

All the LSPR spectra were recorded using 1 cm path length standard quartz cuvettes and a LSPR spectrometer from PerkinElmer, model Lambda650.

### Example 2

### Detection of Biotin Via Streptavidin-Biotin Binding

We detected a biomolecule tagged to biotin using gold nanoparticles (AuNPs) in the gold colloidal suspension. Namely, the binding of an Alexa Fluor® 647 fluorophore tagged to biotin using polyethylene glycol (PEG) to AuNPs surface-modified with streptavidin was detected. This is a proof of concept indicating that any biomolecule tagged to biotin could be similarly detected. Further, when such biomolecules are tagged with fluorophores, the detected biomolecules can be viewed by a fluorescence microscope.

The biosensing protocol was based on a multilayer deposition of different chemical and bio-entities on the AuNPs of the gold colloidal solution (GCS). This deposition was carried out directly in the GCS as follows:
a) The GCS of Example 1 was further diluted with DI water in a quartz cuvette to obtain 0.6 A absorbance units measured using Perkin-Elmer® UV-Vis spectrometer. This approximately corresponds to a gold concentration of 35 μg/mL.
b) Then, 0.5 mL of a 5 mM NanoThinks® ACID11 solution in ethanol was added to the suspension. NanoThinks® ACID11 is a linker that produces a hydrophilic Self-Assembled Monolayer (SAM) on the gold nanoparticles.

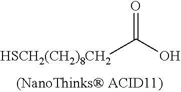

When NanoThinks® ACID11 was added, its thiol groups become chemisorbed on gold nanoparticles by thiolate-Au bond (40-50 kcal/mol) via the adsorption of sulphur. The sulphur is adsorbed on the surface of gold nanoparticles by C—S scission forming a covalent bond.
c) Then, 30 minutes after NanoThinks® ACID11 addition, 0.2 mL of an EDC and NHS mixture was added to the suspension. EDC and NHS solutions with concentrations of 0.1M and 0.05M, respectively, were prepared using PBS and mixed in a 1:1 (v/v) ratio before being added to the suspension.

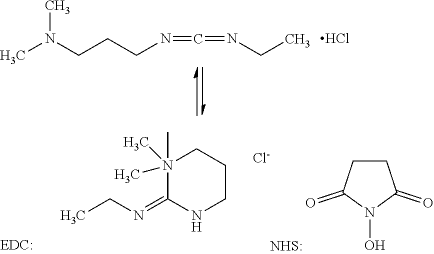

EDC+NHS is a mixture used as a cross linking agent for preparing amine-reactive esters of carboxylate groups for chemical binding and solid-phase immobilization applications. Carboxylates (—COOH) react with NHS in the presence of a carbodiimide such as EDC. Activation with NHS decreases the water solubility of the modified carboxylate molecule. Although, prepared esters are sufficiently stable to process, they will hydrolyze within hours or minutes depending on the water content and the pH of the reaction mixture (NHS esters have a half-life of 4-5 hours at pH 7, 1 hour at pH 8 and only 10 minutes at pH 8.6). EDC reactions are often performed at pH 4.7-6.0 and the best results are obtained when NHS molecules are promptly used to the amine containing targets.
d) Then, 0.2 mL of a streptavidin solution at a concentration of 10 μg/ml was added. Streptavidin is a biotin binding protein, which was originally isolated from Streptomyces avidinii. Streptavidin is mildly acidic with pl of 5 and has no carbohydrate like avidin. Affinity of streptavidin is similar to that of avidin and streptavidin is a tetrameric protein with each subunit binding four biotin molecules. Guanidinium chloride will dissociate avidin and streptavidin into subunits, but streptavidin is more resistant to dissociation.
e) Finally, the thus modified AuNPs were used to bind Biotin-PEG-Alexa647. Thus, a 0.2 mL of Biotin-PEG-Alexa647 solution at a concentration of 20 μg/ml was added to the suspension.

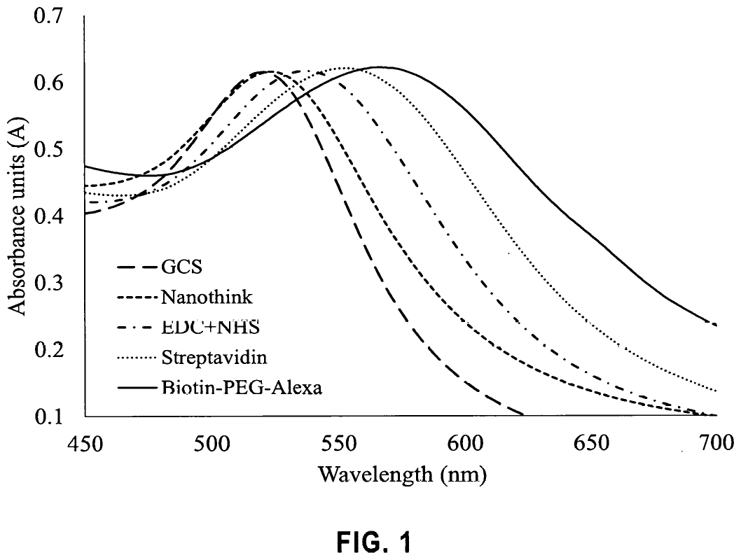

LSPR absorption spectra were recorded at each stage using a Localized Surface Plasmon Resonance (LSPR) spectrometer from PerkinElmer® and the position of the LSPR gold band was recorded. Shifts in the position of this peak were observed after each of the above steps as shown in FIG. 1. In particular, a red shift, observed after addition of biotin-PEG-Alexa647, confirmed the binding of Alexa 647 through biotin to the AuNPs. The shifts in the LSPR band were calculated. Table 1 reports the concentration of the reactant added and the average spectral shift calculated at each step of the protocol.

TABLE 1
Average shift of the Au plasmon band observed
after each step of the biosensing protocol

| | Concentration | | Average |
|---|---|---|---|
| | of reactant | | LSPR shift |
| Step | added | | (Δλ) (nm) |
| NanoThinks ® ACID11 addition | 5 | mM | 3.25 |
| EDC + NHS addition | 0.1M + 0.05M | | 12.5 |
| Streptavidin addition | 10 | μg/ml | 16.25 |
| Biotin-PEG-Alexa647 addition | 20 | μg/ml | 14.25 |

### Example 3

### Detection of Bovine Growth Hormones (BGH) Antigen

We detected bovine growth hormone (BGH) antigen using an AuNPs colloidal suspension via antigen-antibody interactions. By optimizing the concentrations and reaction times of EDC+NHS and the BGH antibody, a sensitivity of <0.1 ng/mL has been achieved.

The biosensing protocol was similar to that described in Example 2, except that after step c) the following steps were performed:
d′) BGH antibody was added to the colloidal suspension and allowed to react and bind to the EDC+NHS; and
e′) then various concentrations of BGH antigen were added to the suspension.

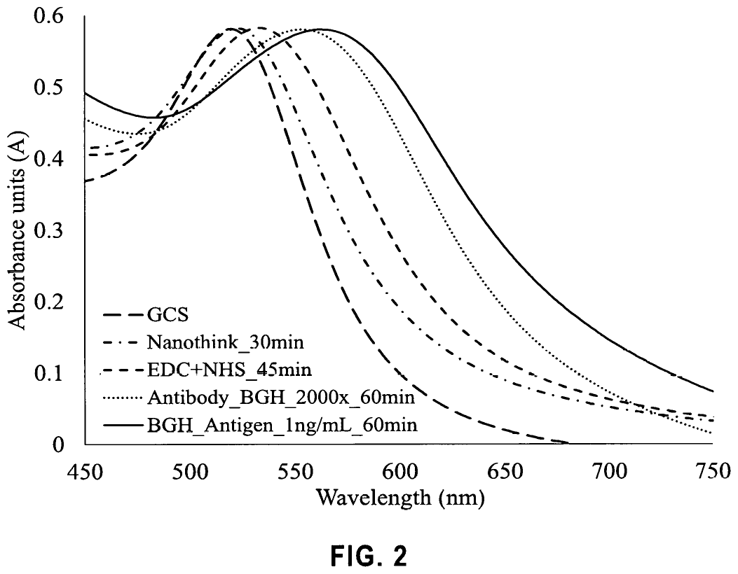

Again, LSPR absorption spectra were measured at each stage of the protocol—see FIG. 2 for representative spectra—and the position of the LSPR gold band was recorded. Furthermore, absorption spectra were measured at different times after BGH antigen addition.

In all cases, a red shift of the LSPR band was observed after BGH antibody addition and then after BGH antigen addition. This confirmed the binding of the BGH antigen to the AuNPs via the BGH antibody.

The shifts in the LSPR band were calculated. Table 2 reports the concentration of the reactant added, how long after reactant addition the spectra were recorded, and the average spectral shift calculated at each step of the protocol.

TABLE 2
Average shift of the Au plasmon band observed
after each step of the biosensing protocol

| | Concentration | | Average |
|---|---|---|---|
| | of reactant | | LSPR shift |
| Step | added | | (Δλ) (nm) |
| NanoThinks ® ACID11_30 | 5 | mM | 3 |
| min after addition | | | |
| EDC + NHS_45 min after addition | 0.1M + 0.05M | | 11 |
| Antibody_60min after addition | 2000x (dilution) | | 19.5 |
| Antigen_60min after addition | 1 | ng/ml | 9 |

Regarding step b), the formation of a self-assembled monolayer of 11-mercaptoundecanoic acid (NanoThinks® ACID11, concentration of 5 mM) with a standard LSPR shift around 4 nm was achieved within 30 minutes.

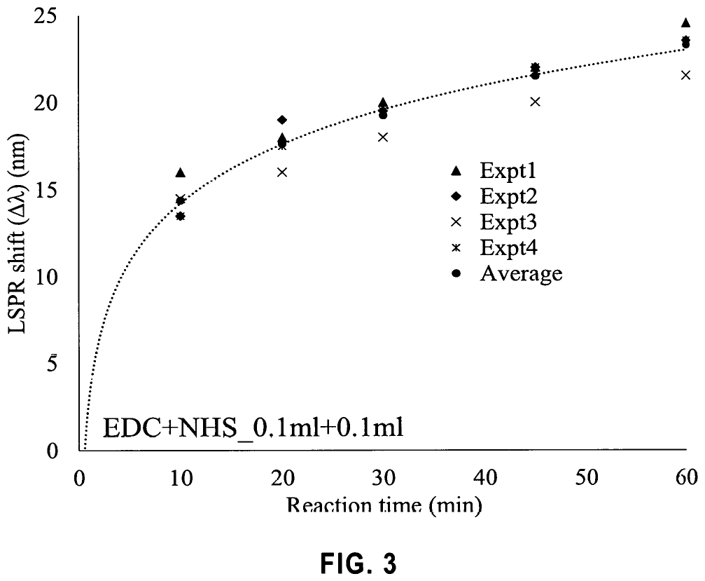

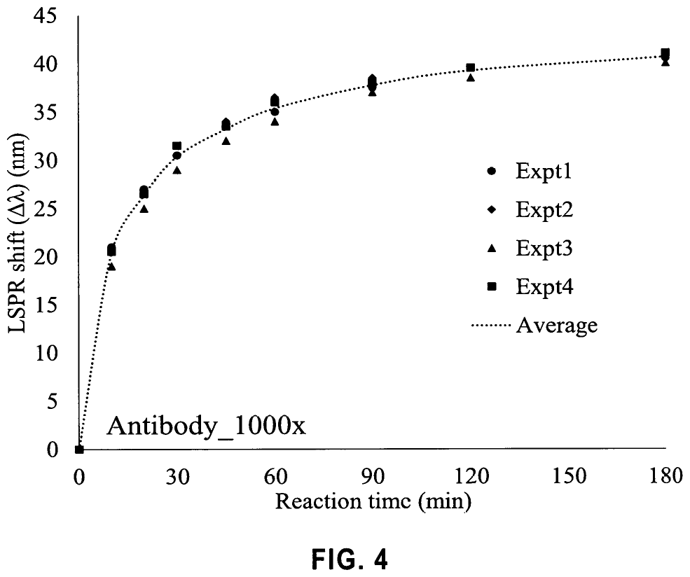

On the other hand, the reaction times of steps c) and d′) were optimized to achieve maximum molecule binding to the AuNPs. To do so, the LSPR absorption spectra were measured at different intervals after reactant(s) addition. Maximal molecule binding was deemed to be achieved when the LSPR gold shift reached a plateau. FIGS. 3 and 4 show the evolution of the LSPR shift with reaction time after EDC+NHS addition (FIG. 3) and BGH antibody addition (FIG. 4). The binding of EDC+NHS almost reached a plateau after 45 minutes, while the binding of BGH antibody had reached its maximum after 1 h.

### Example 4

### Detection and Isolation of Exosomes

We detected and isolated exosomes from the MCF7 breast cancer cell line media.

The biosensing protocol was similar to that described in Example 2, except that after step d) the following steps were performed:

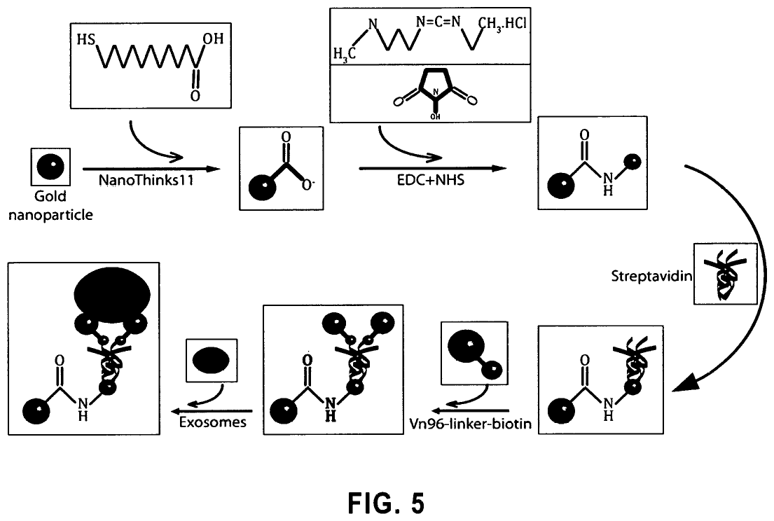

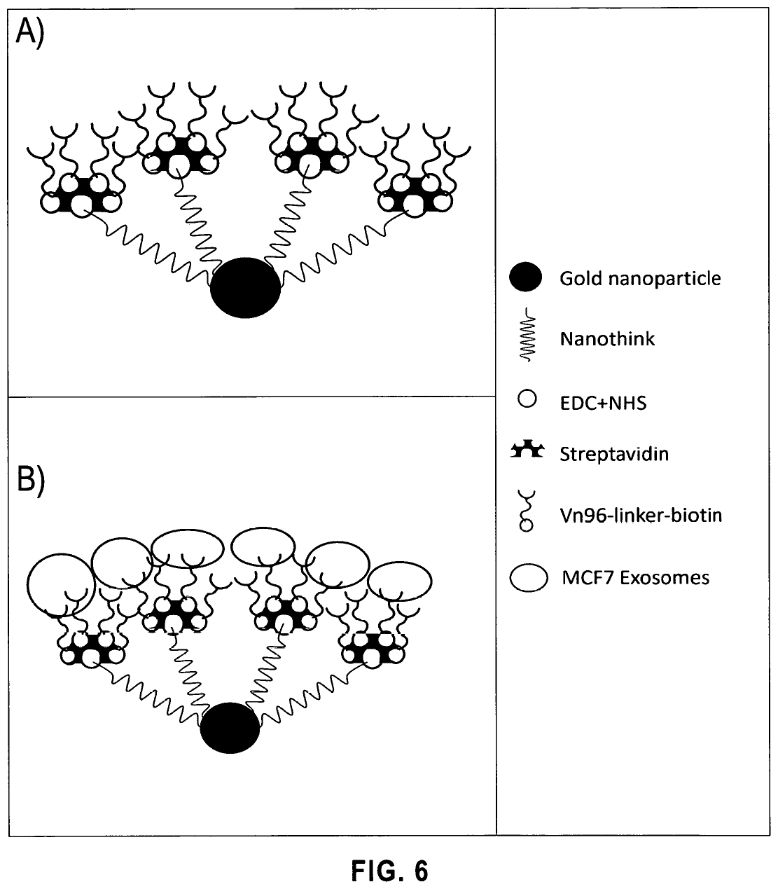

e″) 0.2 mL of Vn96-linker-biotin with a concentration of 26 μg/mL was added to the colloidal suspension. Vn96 is a synthetic peptide that was specifically designed to capture exosomes by binding to the heat shock proteins (HSP) present on their surface. In this protocol, Vn96 was linked to biotin so it could become attached to the AuNPs via attachment to streptavidin. The linker was 6-aminohexanoic acid (NH₂—(CH₂)₅—COOH).
f′) Then, 0.2 mL of various concentrations of Exosomes (undiluted and diluted 5, 10, 20 and 25 times, the exosomes concentration of the undiluted exosomes being 1.19×10¹² particles/mL) were added to the suspension.
   This protocol is shown in FIG. 5, with steps e″ and f″ shown in more details in FIG. 6.

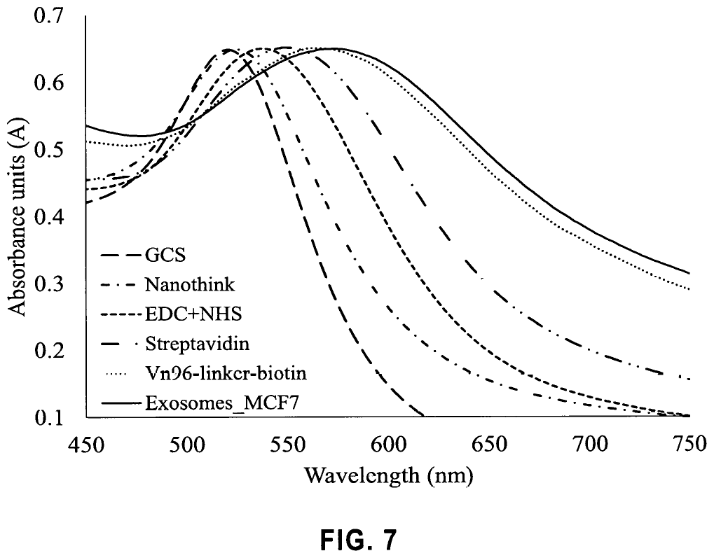

In a typical experiment, the LSPR absorption spectrum of the gold colloidal suspension (at step a) was first measured. Then, LSPR spectra were measured after each protocol step and the position of the LSPR gold band was recorded. The shift in the position of this band confirmed successful binding at each step. Representative absorption spectra are shown in Erreur ! Source du renvoi introuvable. FIG. 7.

The reactant concentrations and reaction times were optimized for each step in the manner described in Example 3.

For comparison, a similar biosensing protocol was carried out using gold immobilized on a glass substrate rather than an AuNPs colloidal suspension. The gold immobilized on the glass substrate was prepared as described in Bathini et al. Nano-Bio Interactions of Extracellular Vesicles with Gold Nanoislands for Early Cancer Diagnosis, Research, 2018, Article ID 3917986, 10 pages.

Table 3 shows the spectral shifts observed at each step of the protocol using the AuNPs colloidal suspension and the immobilized gold. The shift observed with the AuNPs colloidal suspension was up to almost five times larger than that observed when the immobilized gold is used.

TABLE 3
Shift of the Au plasmon bands corresponding
to the different steps of the protocol

| | | | | | Δλ (nm) | Δλ (nm) |
|---|---|---|---|---|---|---|
| | Original | | Optimized | | (Gold on | (Colloidal |
| Entity | Concentration | | Concentration | | Glass) | Suspension) |
| 11-MUA* | | | 5 | mM | 5 ± 0.5 | 4.62 ± 0.3 |
| EDC + NHS | | | 0.1M + 0.05M | | 4.82 ± 0.3 | 12.43 ± 1.5 |
| Streptavidin | 1 | mg/mL | 10 | μg/mL | 3.08 ± 1 | 17.82 ± 4 |
| Vn96-linker- | 1.3 | mg/mL | 13 | μg/mL | 5.67 ± 1.5 | 17.6 ± 3 |
| biotin | | | | | | |

| | | | | |
|---|---|---|---|---|
| Exosomes | 1.19 × 10¹²/mL | 2.38 × 10¹¹/mL | 4 ± 1 | 7.25 ± 2 |
| (MCF7)** | | | | |

*11-Mercaptoundecanoic acid
**Measured before sedimentation, which is slower with lower Vn96-linker-biotin concentrations (such as13 μg/mL).

### 4A) Sedimentation and Isolation of Exosomes

When Vn96-linker-biotin was added after the streptavidin stage, flocculation (with the floc floating on top of the liquid) was observed. This is probably be due to the low self-weight when Vn96 molecules bind to the streptavidin through the biotin. In some case, precipitation was noticed at the Vn96 stage, but only when the streptavidin and Vn96 concentrations were relatively high.

However, when MCF7 exosomes were added to the suspension, there was a noticeable circular movement of the colloids in the suspension and the floc gradually settled down yielding a precipitate and a supernatant. This made exosome isolation possible. The sedimentation caused by the colloidal gold solution was quick and easy to collect for liquid biopsy

Indeed, the number of molecular interactions between the AuNPs and the exosomes depends on the gold concentration and the number of available exosomes. Ideally, all the AuNPs and all the exosomes become bound together and settle as a precipitate and there are no exosomes or AuNPs left in the supernatant. In this ideal case, the LSPR absorption spectrum of the supernatant would be that of water. When the AuNPs and exosomes concentrations are mismatched, either only AuNPs or only exosomes should be observed in the LSPR absorption spectrum of the supernatant.

Here, however, when the LSPR absorption spectrum of the supernatant was measured, two peaks at about 265 nm and 570 nm (belonging to proteins and gold respectively) were observed. We subsequently established that the protein peak did not arise from exosomes in the supernatant, but rather other proteins.

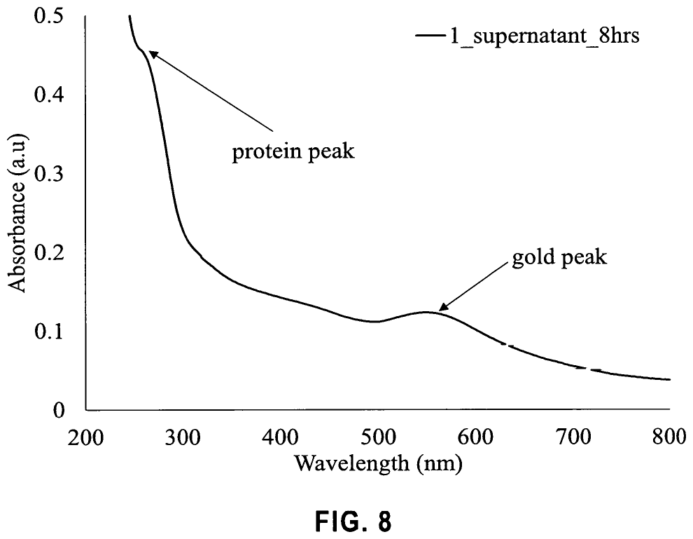

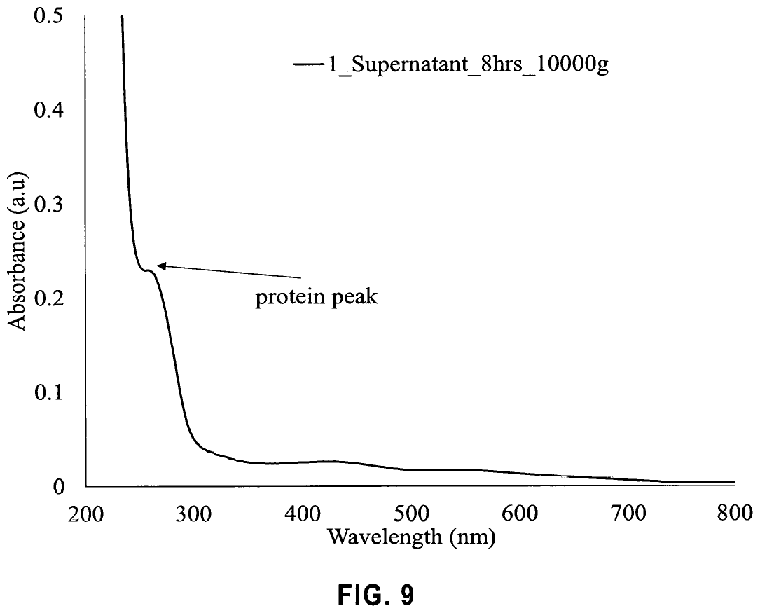

When the supernatant consisting of protein and gold was centrifuged at 10,000 g for 2 minutes, the gold peak disappeared from its LSPR absorption spectrum, leaving the protein peak almost unchanged. Absorption spectra of the supernatant before and after the centrifugation is shown in FIGS. 8 and 9. It can be concluded, from the spectrum recorded after centrifugation, that the small peak at about 265 nm is not due to exosomes (which would have settled upon centrifugation) but rather other protein(s)—possibly arising from the culture media.

Therefore, the gold concentration used was sufficient to isolate all exosomes from the suspension.

### 4B) Tuning AuNPs Concentration

We noted in the previous section that to isolate all exosomes via precipitation, an AuNP concentration sufficient to capture all the exosomes must be present. In other words, when low concentrations of exosomes are present, lower concentration of AuNPs can be used while higher exosomes concentrations require higher AuNPs concentration. Thus, the AuNPs concentration can be tuned as needed for example selecting a concentration that would isolate all exosomes from a “normal” sample, while not being enough to isolate all the exosomes found in a cancerous sample.

This is simply achieved by recording the LSPR absorption spectrum of the supernatant after precipitation and check whether the gold band is still present (as an absence of this band would indicate that all the gold was used). We show below that the gold concentration in the suspension can be successfully tuned:
by varying the concentration of gold precursor (HAuCl₄.3H₂O) used to prepare the suspension or
by diluting the suspension with DI water.

Suspensions with various concentrations of AuNPs were prepared. Gold colloidal suspension was prepared using 23 mg of as described in Example 1. To assess reproducibility of the method, gold colloidal suspensions were prepared as described in Example 1 but using higher and lower quantities (18 mg and 30 mg) of HAuCl₄.3H₂O.

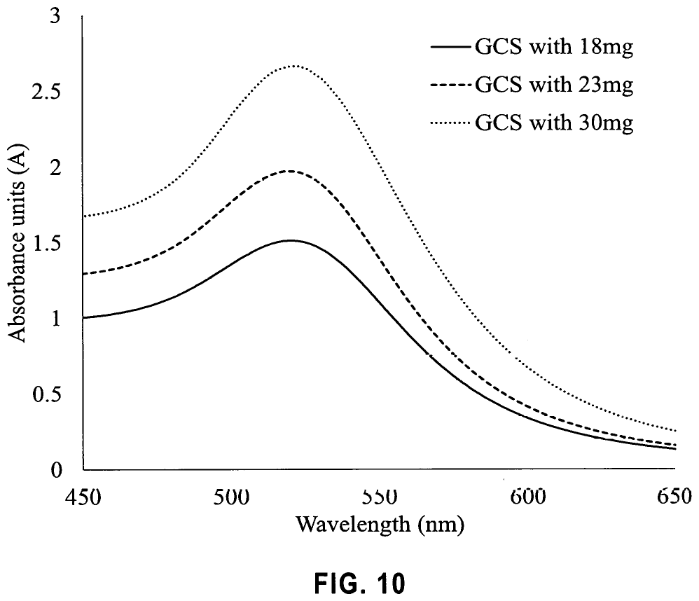

The LSPR absorption spectra of the thus produced GCSs are shown in FIG. 10. As expected, the more concentrated the suspension, the higher the absorbance of the gold band.

All suspensions were diluted with DI water to reach various gold concentrations. LSPR was used to make sure that, at each desired concentration, all of the diluted suspensions had the same concentration. Namely, for each desired concentration, the suspensions were diluted until their LSPR gold band had the same intensity, i.e. 0.25, 0.5, or 1 a.u.

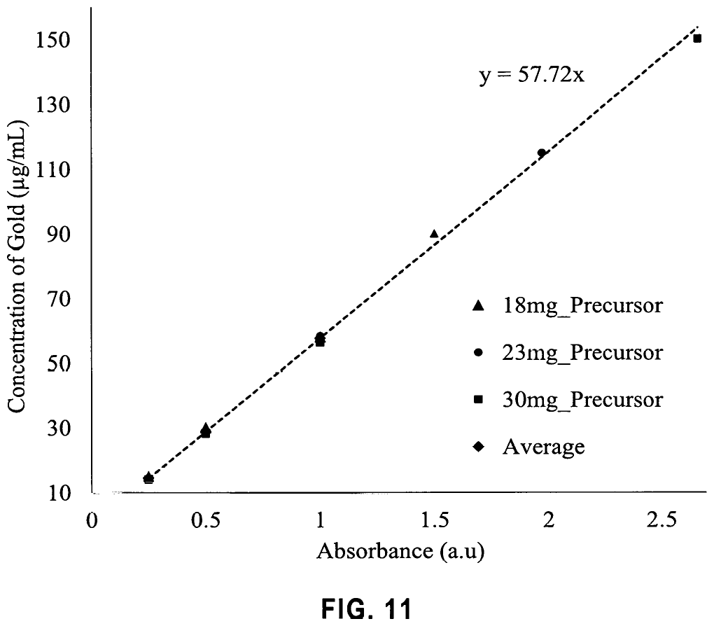

The gold concentration of the supernatant undiluted and diluted suspensions as function of their LSPR gold band absorbance is shown in FIG. 11.

The protocol described above was then carried out using these suspensions. The suspensions sedimented after exosome addition. The solutions in the cuvettes remained untouched until the precipitate settled down, which took about 4 hours. Once the precipitate settled, the supernatant was isolated from the cuvette, using a syringe and its LSPR absorption spectrum was measured.

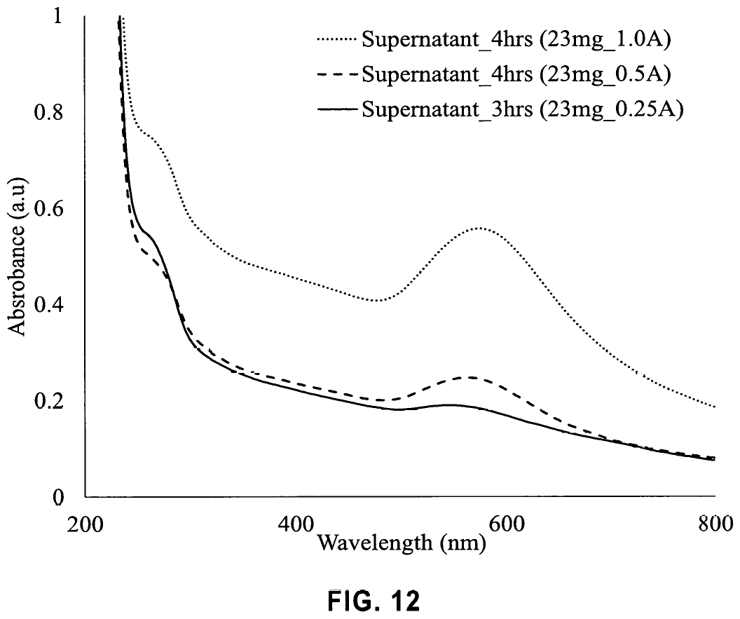

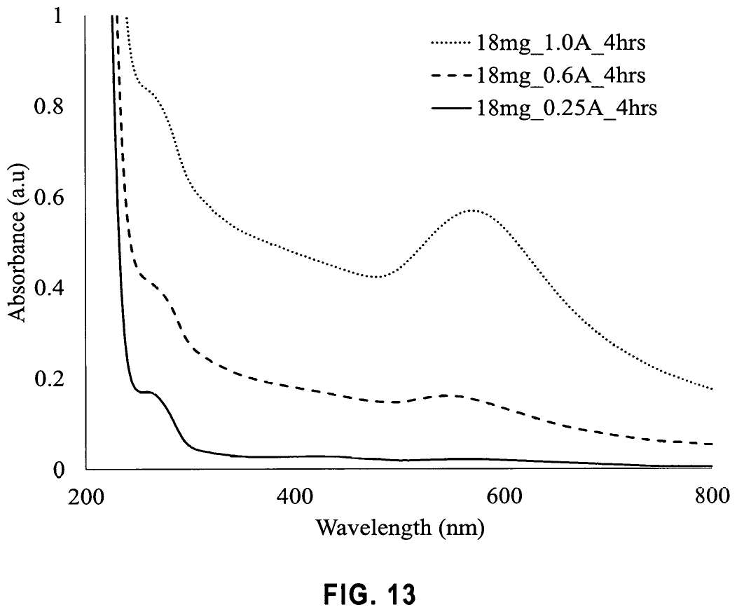

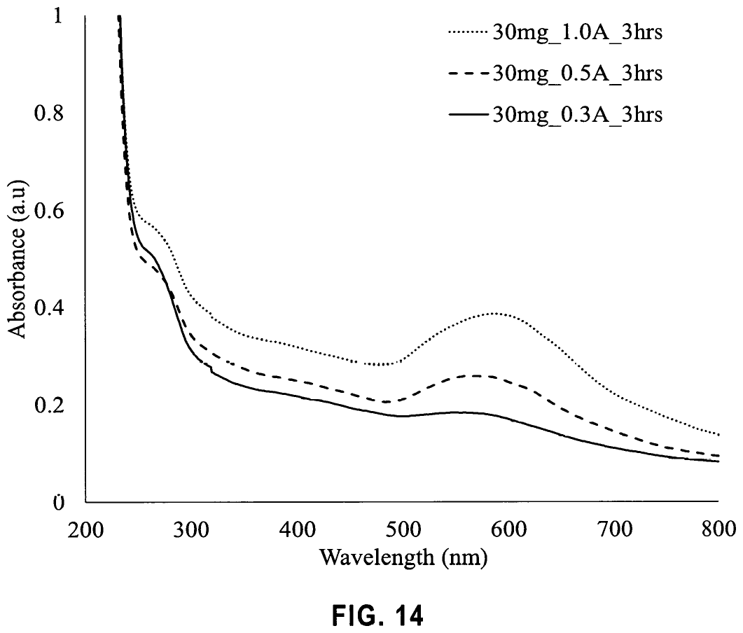

The measured absorption spectra for the various diluted suspensions are shown in FIGS. 12, 13, and 14. In each case, they showed peaks at around 265 nm and around 570 nm. The first peak is the same small protein peak discussed above. The peak at 570 nm corresponds to the gold nanoparticles. It can be observed that the intensity of the gold peak, which corresponds to the concentration of “extra” gold remaining in suspension after precipitation, decreases with decreasing initial gold concentration. We conclude that all exosomes were removed from the suspension. If extra exosomes had remained in the supernatant, the protein peak would have been bigger, and the gold peak would have been absent. It can be seen from FIG. 13 that the supernatant spectrum for 18 mg with an absorbance value of 0.25 had a very small or almost absent gold peak, meaning that no extra gold nanoparticles were present in the supernatant after precipitation.

### 4C) Using the Supernatant as a Biosensor

As noted above, the LSPR absorption spectrum of the supernatant obtained after precipitation provides information as to how much exosomes were added to the suspension:
If a gold peak is observed, the gold concentration was enough to remove all exosomes. In other words, the exosome concentration did not exceed the gold removal capacity.
If a strong protein peak is observed and no gold peak is observed, the gold concentration could not remove all exosomes. In other words, the exosome concentration did exceed the gold removal capacity.
   This gold removal capacity is the cut-off exosome concentration above which the suspension can no longer remove exosome. This gold removal capacity can be calculated using the AuNPs concentration as well as the size of the AuNPs and the size of the exosomes.

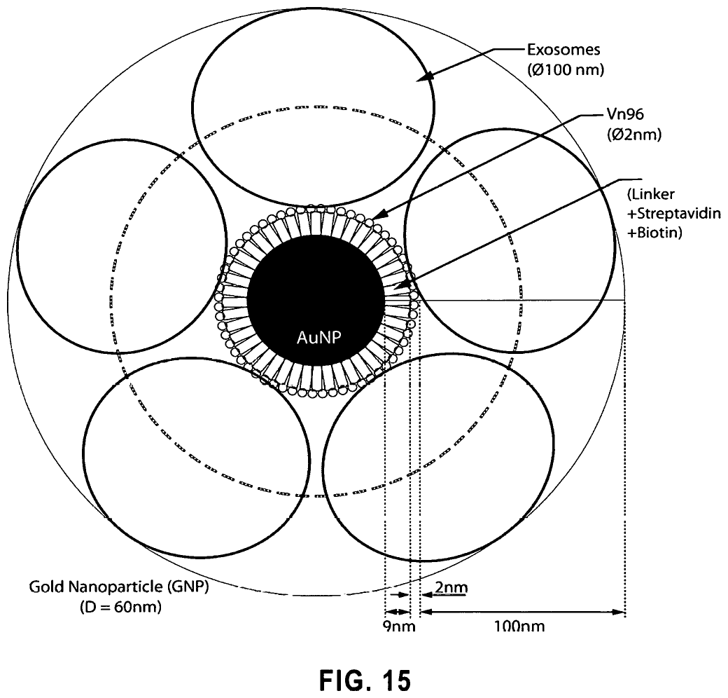

Indeed, the number of exosomes that can be accommodated on each AuNPs depends on the nanoparticle diameter and the exosome diameter. This number can be calculated using the surface area of the AuNPs—including all molecules attached thereon (ligands, streptavidin and Vn96)—and the surface area of the exosomes as shown in FIG. 15. The number of AuNPs in the suspension can be calculated from the AuNPs concentration, which provides the total mass of the AuNPs in the suspension, and the size of the AuNPs, which provides their individual weight. Finally, knowing the number of AuNPs and the number of exosomes that can be accommodated by each AuNPs, the total number of exosomes that can be removed from the suspension by a given suspension can easily be calculated.

In the present case, the size distribution of the exosomes/particles in the culture media of MCF7 cell line was measured using a Tunable Resistive Pulse Sensing (TRPS) measurements (qNano® from iZON® science) and found to be 1.33×10¹⁰ particles/mL. MCF7 is a breast cancer cell line so this concentration can be considered here as representative of a “cancerous sample” An exosome concentration 50 times smaller (50×dilution) of this cancerous sample can be considered here as representative of a “non-cancerous sample”—see C. Chen et al., “Microfluidic isolation and transcriptome analysis of serum microvesicles,” Lab Chip, vol. 10, pp. 505-511, 2009, incorporated herein by reference.

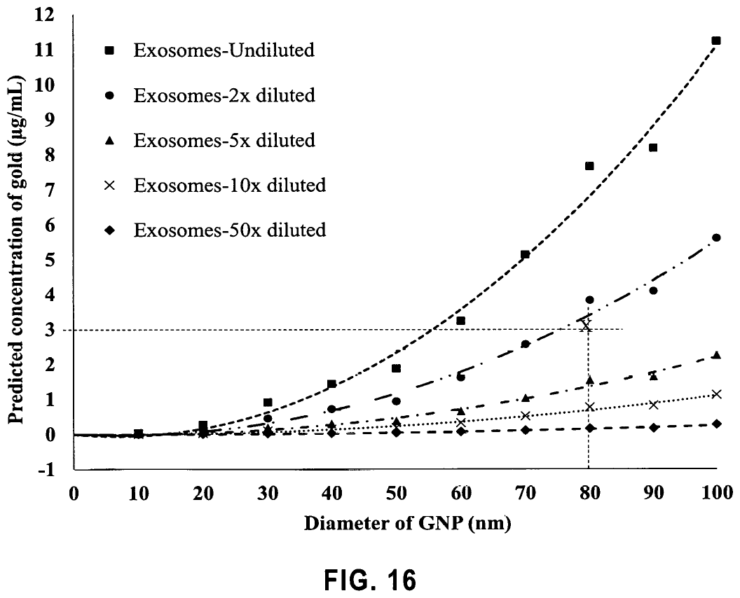

FIG. 16 is a plot of the predicted gold concentration required to remove all the exosomes of the above representative cancerous sample (labelled “Undiluted”) and this sample diluted by a factor of 2, 5, 10, or 50 (the latter corresponding the above representative non-cancerous sample as a function of AuNPs size (alone, without considering the attached molecules). The area between the “cancerous” and “non-cancerous” curves contains the parameter combinations (AuNPs size and concentration) that can be used for discriminating between cancerous and non-cancerous samples. For example, the point labelled with an “X” identified one such gold colloidal suspension comprising with nanoparticles of 80 nm in diameter and a gold concentration of 3 μg/mL.

If such a gold colloidal suspension were used with an unknown exosome-containing sample. The sample would be added to the suspension, a precipitate would form, the LSPR absorption spectrum of the supernatant would be recorded. If this spectrum showed a gold peak, it would mean that all the exosomes had been captured and settled along with the precipitate, which would indicate that the sample as a non-cancerous sample. If a strong protein peak was observed, it would mean that excess exosomes had not been captured/settled, which would indicate that the sample as a cancerous sample.

### REFERENCES

The present description refers to a number of documents, the content of which is herein incorporated by reference in their entirety. These documents include, but are not limited to, the following:
Bathini et al. Nano-Bio Interactions of Extracellular Vesicles with Gold Nanoislands for Early Cancer Diagnosis, Research, 2018, Article ID 3917986, 10 pages
Di Noto et al. Merging colloidal nanoplasmonics and surface plasmon resonance spectroscopy for enhanced profiling of multiple myeloma-derived exosomes, Biosens Bioelectron. 2016 Mar. 15; 77: 518-24
Gao et al., A dual signal amplification method for exosome detection based on DNA dendrimer self-assembly, Analyst, 2019,144, 1995-2002
Jiang et al. Aptamer/AuNP Biosensor for Colorimetric Profiling of Exosomal Proteins, Angewandte Chemie, International Edition, 2017, 56, 39, 11916-11920
Roma-Rodrigues et al, Tumor Microenvironment Modulation via Gold Nanoparticles Targeting Malicious Exosomes: Implications for Cancer Diagnostics and Therapy, Int. J. Mol. Sci. 2017, 18(1), 162
Wang et al. Recent Progress in Isolation and Detection of Extracellular Vesicles for Cancer Diagnostics, Advanced Healthcare Materials, 2019, ID 1800484
N. Uehara, “Polymer-functionalized Gold Nanoparticles as Versatile Sensing Materials,” Anal. Sci., vol. 26, pp. 1210-1228, 2010.
C. Chen et al., “Microfluidic isolation and transcriptome analysis of serum microvesicles,” Lab Chip, vol. 10, pp. 505-511, 2009.
L. Dykman and N. Khlebtsov, “Chem Soc Rev Gold nanoparticles in biomedical applications: recent advances and perspectives to biological and medical,” Chem Soc Rev, vol. 41, pp. 2256-2282, 2012.
G. Fuhrmann, I. K. Herrmann, and M. M. Stevens, “Cell-derived vesicles for drug therapy and diagnostics: opportunities and challenges.,” Nano Today, vol. 10, no. 3, pp. 397-409, June 2015.
B. György et al., “Membrane vesicles, current state-of-the-art: emerging role of extracellular vesicles.,” Cell. Mol. Life Sci., vol. 68, no. 16, pp. 2667-88, August 2011.
M. Yáñez-Mó et al., “Biological properties of extracellular vesicles and their physiological functions,” J. Extracell. Vesicles, 2015.
E. van der Pol, A. N. Böing, P. Harrison, A. Sturk, R. Nieuwland, and B. Engineering, “Classification, Functions, and Clinical Relevance of Extracellular Vesicles,” Pharmacol. Rev., vol. 64, pp. 676-705, 2012.
J. Conde-Vancells et al., “Characterization and comprehensive proteome profiling of exosomes secreted by hepatocytes,” J. Proteome Res., vol. 7, no. 12, pp. 5157-66, December 2008.
M. He, J. Crow, M. Roth, Y. Zeng, and A. K. Godwin, “From chip-in-a-lab to lab-on-a-chip: towards a single handheld electronic system for multiple application-specific lab-on-a-chip (ASLOC),” Lab Chip, vol. 14, p. 3773, 2014.
S. Singh Kanwar, A. Christopher, J. Dunlay, D. M. Simeone Cde, and S. Nagrath, “Lab on a Chip Microfluidic device (ExoChip) for on-chip isolation, quantification and characterization of circulating exosomes,” Lab Chip, vol. 14, p. 1891, 2014.
S. Yong Zeng, Z. Zhao, A. Yang Yang, Y. Zeng, and M. He, “A microfluidic ExoSearch chip for multiplexed exosome detection towards blood-based ovarian cancer diagnosis As featured in: A microfluidic ExoSearch chip for multiplexed exosome detection towards blood-based ovarian cancer diagnosis,” Lab Chip, vol. 16, p. 489, 2016.
I. Sarkar and A. K. Mishra, “Fluorophore tagged bio-molecules and their applications: A brief review,” Appl. Spectrosc. Rev., vol. 53, no. 7, pp. 552-601, 2018.
H. Sahoo, “Fluorescent labeling techniques in biomolecules: a flashback,” RSC Adv., vol. 2, pp. 7017-7029, 2012.
T. T. Weil, R. M. Parton, and I. Davis, “Making the message clear: visualizing mRNA localization,” Trends Cell Biol., vol. 20, no. 7, pp. 380-90, July 2010.
J. Ozhikandathil, S. Badilescu. and M. Packirisamy, “Gold nanoisland structures integrated in a lab-on-a-chip for plasmonic detection of bovine growth hormone,” J. Biomed. Opt., vol. 17, no. 7, p. 0770011, 2012.
J. Ozhikandathil, S. Badilescu, and M. Packirisamy, “Detection of fluorophore-tagged recombinant bovine somatotropin (rbST) by using a silica-on-silicon (SOS)-PDMS lab-on-a-chip,” IEEE Sens. J., vol. 12, no. 9, pp. 2791-2798, 2012.
C. SIM, Sang Jun, KIM, Jun Pyo, “Method of detecting bioproducts using localized surface plasmon resonance sensor of gold nanoparticles,” Patent, WO 2009/1021192 Al (72), 2009.
J. Kimling, M. Maier, B. Okenve, V. Kotaidis, H. Ballot, and A. Plech, “Turkevich Method for Gold Nanoparticle Synthesis Revisited”, J. Phys. Chem. B 2006, 110, 15700-15707.
Turkevich, J.; Stevenson, P. C.; Hillier, J. Discuss. Faraday Soc. 1951, 11, 55; Enu\ddot{ }stu\ddot{ }n, B. V.; Turkevich, J. J. Am. Chem. Soc. 1963, 85, 3317.
CN 102590521 A
CN 103940992A
CN 103954773A
CN 105527447A
CN 107132209A
US 2002/0164611; US 2014/0242613
WO 2004/042084; WO 2018/011474

1. A method of quantifying an analyte in a sample, the method comprising the steps of:
a) providing a colloidal suspension of nanoparticles of a plasmonic metal, the nanoparticles having attached on their surface a binding moiety for selective attachment of said analyte,
b) adding the sample to the suspension, thus producing a mixture in which said analyte is attached to the nanoparticles in suspension,
c) allowing sedimentation of the nanoparticles with bound analyte, thereby producing a sediment comprising the nanoparticles with bound analyte and a supernatant, and
d) measuring the Localized Surface Plasmon Resonance (LSPR) spectrum of the supernatant,
wherein the colloidal suspension comprises a known concentration of nanoparticles which can bind a known cut-off concentration of the analyte,
wherein the presence of a plasmonic metal band in the LSPR spectrum of the supernatant indicates the sample had a concentration of the analyte lower than said cut-off concentration,
wherein the presence of band associated with the analyte in the LSPR spectrum of the supernatant indicates that the sample had a concentration of the analyte higher than said cut-off concentration, and
wherein the analyte is a biomolecule or a vesicle.

2. A method of diagnosing a condition characterized by an excess or a depletion of an analyte in a biological sample, the method comprising steps of:
a) providing a colloidal suspension of nanoparticles of a plasmonic metal, the nanoparticles having attached on their surface a binding moiety for selective attachment of said analyte,
b) adding the sample to the suspension, thus producing a mixture in which said analyte is attached to the nanoparticles in suspension,
c) allowing sedimentation of the nanoparticles with bound analyte, thereby producing a sediment comprising the nanoparticles with bound analyte and a supernatant, and
d) measuring the Localized Surface Plasmon Resonance (LSPR) spectrum of the supernatant,
wherein the colloidal suspension comprises a known concentration of nanoparticles which can bind a known cut-off concentration of the analyte, wherein said cut-off concentration is between the average concentration of the analyte found in healthy subjects and the average concentration of the analyte found in subjects suffering from said condition,
wherein the presence of a plasmonic metal band in the LSPR spectrum of the supernatant indicates that the sample had a concentration of the analyte lower than said cut-off concentration,
wherein the presence of band associated with the analyte in the LSPR spectrum of the supernatant indicates that the sample had a concentration of the analyte higher than said cut-off concentration, and
wherein the analyte is a biomolecule or a vesicle.

3. The method of claim 1 or 2, wherein centrifugation is used at step c) to speed up the sedimentation of the nanoparticles.

4. A method of isolating analyte from a sample, the method comprising the steps of:
a) providing a colloidal suspension of nanoparticles of a plasmonic metal, the nanoparticles having attached on their surface a binding moiety for selective attachment of said analyte;
b) adding the sample to the suspension, thus producing a mixture in which said analyte is attached to the nanoparticles in suspension;
c) allowing sedimentation of the nanoparticles with bound analyte, thereby producing a sediment comprising the nanoparticles with bound analyte and a supernatant;
d′) recovering the sediment,
wherein the analyte is a biomolecule or a vesicle.

5. The method of claim 4, further comprising the steps of:
optionally, recovering the analyte form the sediment, and
carrying out molecular analysis on the sediment or on the separated analyte.

6. A method of detecting an analyte in a sample, the method comprising the steps of:
a) providing a colloidal suspension of nanoparticles of a plasmonic metal, the nanoparticles having attached on their surface a binding moiety for selective attachment of said analyte;
b) adding the sample to the suspension thus producing a mixture in which said analyte is attached to the nanoparticles in suspension; and
c′) measuring the Localized Surface Plasmon Resonance (LSPR) spectrum of the mixture,
wherein a shift in the position of the plasmonic metal band in the LSPR spectrum of the mixture compared to that of the suspension before addition of the sample indicates the presence of said analyte in the sample, and
wherein the analyte is a biomolecule or a vesicle.

7. The method of any one of claims 1 to 6, wherein the analyte is a biomolecule or a vesicle, preferably a vesicle, and more preferably an exosome.

8. The method of any one of claims 1 to 7, wherein the sample is a biological sample, preferably a biofluids.

9. The method of any one of claims 1 to 8, further comprise, before step a), a step of preparing nanoparticles of a plasmonic metal, the nanoparticles having attached on their surface a binding moiety for attachment of said analyte.

10. The method of any one of claims 1 to 9, wherein the binding moiety is attached to a linker, which is attached to the nanoparticle surface.

11. A device for performing the method of any one of claims 1 to 10, the device comprising:
a) a suspension inlet (12) for introducing a colloidal suspension of nanoparticles of a plasmonic metal, the nanoparticles having attached on their surface a binding moiety for selective attachment of said analyte,
b) a sample inlet (14) for introducing the sample into the device,
c) a mixing chamber (16) for mixing the sample with the suspension, thus producing a mixture in which said analyte is attached to the nanoparticles in suspension, and
d) a sedimentation network (18) to allow sedimentation of the nanoparticles with bound analyte, thereby producing a sediment comprising the nanoparticles with bound analyte and a supernatant,
wherein the sedimentation network (18) comprises a sediment container (20) and a supernatant container (22).

12. The device of claim 11, wherein the sedimentation network (18) is gravity-assisted.

13. The device of claim 11 or 12, wherein the sedimentation network (18) comprises a serpentine microfluidic channel.

14. The device of any one of claims 11 to 13, wherein the supernatant container (22) comprises a sediment outlet (24) to allow recovery of the sediment.

15. The device of any one of claims 11 to 14, wherein the sedimentation network (18) comprises a supernatant outlet (26) to allow recovery of the supernatant for LSPR analysis.

16. The device of any one of claims 11 to 14, wherein the supernatant container (22) functions as a cuvette allowing recording the LSPR spectrum while the supernatant is held in the device.

17. The device of any one of claims 11 to 16, wherein the device (10) is a hand-held device.

18. The device of any one of claims 11 to 16, wherein the device (10) is a macroscale device, a microscale device, or in a microfluidic device.

19. The device of any one of claims 15 to 18, wherein the device (10) is a point-of-care testing device e.g. for quantifying an analyte in a sample and/or diagnosing a condition characterized by an excess or a depletion of an analyte in a biological sample, e.g. cancer.

20. The device of any one of claims 15 to 19, wherein the device (10) is for rBGH detection.

21. The device of any one of claims 15 to 20, wherein the device (10) comprises multiple isolation units (i.e. inlets (12, 14), mixing chamber (16), and sedimentation network (18)) allowing the detection of analyte in various samples.
